(19) United States
(12) Patent Application Publication
(10) Pub. No.: US 2006/0293568 A1
(43) Pub. Date: Dec. 28, 2006

(54) Collapsible surgical tool bar

(76) Inventor: Rosemarie Sonja Scheidegger-Pluss, Mellingen/Aargau (CH)

Correspondence Address: WINSTON & STRAWN LLP;PATENT DEPARTMENT, 1700 K STREET, N.W., WASHINGTON, DC, 20006, US

(21) Appl. No.: 11/166,170

(22) Filed: Jun. 27, 2005

(51) Int. Cl. A61B 1/32

(52) U.S. Cl. 600/227

(57) ABSTRACT

A surgical tool support that includes a first elongated portion and a second elongated portion in supportive association with the first elongated portion and extending therefrom at a first angle of less than 180°. The second elongated portion is configured for adjustably and fixably being secured to a base member for supporting the tool support. The tool support also includes a first section, which includes the first elongated portion, and a second section, which includes at least part of the second elongated portion. A movable joint movably connects the first and second sections of the tool support for movement between an operative position and a compact position, in which the first and second sections are oriented such that the tool support is substantially more compact in the compact position than in the operative position. A locking member is also associated with the movable joint for releasably locking the movable joint in the operative position with sufficient strength and rigidity such that first elongated portion and any surgical tool mounted thereto is substantially rigidly supported by the second section.

### FIELD OF THE INVENTION

The present invention relates generally to a device for assisting physicians during surgical procedures. More particularly, the invention relates to a surgical tool support device for supporting surgical instruments during surgical procedures.

### BACKGROUND OF THE INVENTION

Surgical tool support devices exist to assist physicians as they perform surgical procedures on patients. These devices typically include one or more fixed and pivotable arms that are secured to an operating room table and that support a surgical tool or instrument mounted to the arms. One surgical tool rod is described in U.S. Pat. No. 4,143,652, the content of which is hereby incorporated herein by reference thereto, and which is sold under the trademark Iron Intern® by Automated Medical Products Corp.

U.S. Pat. No. 5,224,680 discloses another type of surgical instrument holder that is adapted to be received and retained by an operating room table clamping apparatus. The device is configured to adjustably mount and stabilize a surgical instrument, such as a laparoscope or endoscope, in a desired position over a patient, thus lessening the number of personnel that is required to assist during the surgical procedure. The surgical instrument holder is particularly useful during laparoscopic procedures, where it is necessary to routinely remove the laparoscope in order to clean or defog the lens thereof and then return and reposition the instrument in substantially the exact position within the patient to continue viewing the targeted area. The surgical instrument holder provides sufficient rigidity to prevent slippage or inadvertent movement, which could lead to loss of position when a laparoscope is replaced after cleaning, or when a surgical retractor is held under a force, such as elevated when supporting the rib cage of an obese patient.

L-shaped tool support devices often include a vertical portion that has sufficient height so that a horizontal portion extends therefrom to mount surgical instruments or a horizontal cross-bar in a convenient position over the patient. Some devices, such as those manufactured by Thompson Surgical Instruments, Inc., include an adjustable clamp or retaining feature that allows a portion of the device to be positioned in an infinitely adjustable manner, including in a horizontal position. By incorporating an adjustable clamp, however, the tool support device cannot reliably and repeatably be positioned precisely, requiring the surgical team to carefully perform an extra adjustment when setting up the support. It is desirable, however, to minimize the adjustments of equipment that need to be performed during the surgery.

Trays for the sterilization of surgical instruments are made in various sizes and are used for placing the instruments in an autoclave. In the United States, typical sterilization trays have a width of 9 inches, a length or 26 inches, and a height of 3.5 inches, or a width of 8 inches, a length of 25.25 inches, and a height of 3.5 inches. Another typical sterilization procedure is to wrap the device in two sheets to be placed in the autoclave. The outer sheet is removed outside the sterile field in the operating room, and the inner sheet is opened within the sterile field adjacent the patient.

PCT Publication No. PCT/US00/04696 discloses a tool support device that includes a horizontal bar that can support surgical tools therefrom and that can also fit within conventional sterilization trays. The horizontal bar is formed of two or more pieces that are screwed or otherwise attached together. When detached, each of the pieces is short enough to fit within a conventional sterilization tray and when assembled, the bar is long enough for practical use during surgical procedures. It is desirable to reduce the number of pieces of a device to decrease the risk that any single piece may be misplaced or that the assembly will be incorrect. Also, because each piece needs to be accounted for in the operating room, a device that incorporates less detachable pieces is easier and more practical to use.

Thus, there is a need for a practical and useful tool support device that can fit within a standard sterilization tray to allow sterilization of the device, while also having a minimum number of separable pieces being sufficiently rigid and tough to support the tools.

### SUMMARY OF THE INVENTION

The present invention relates to a surgical tool support device. The preferred embodiment includes a first elongated portion comprising a tool mount that is configured to mount a surgical tool thereto. The device also includes a second elongated portion in supportive association with the first elongated portion and extending therefrom at a first angle of less than 180°. The first angle is preferably between about 70° and 110°. The second elongated portion is configured for adjustably and fixably being secured to a base member for supporting the tool support, wherein the tool support comprises a first section, which includes the first elongated portion, and a second section, which includes at least part of the second elongated portion. The device further includes a moveable joint that movably connects the first and second sections of the tool support for movement between an operative position and a compact position, in which the first and second sections are oriented such that the tool support is substantially more compact than in the operative position. Additionally, the device includes a locking member associated with the movable joint for releasably locking the movable joint in the operative position with sufficient strength and rigidity such that first elongated portion and any surgical tool mounted thereto is substantially rigidly supported by the second section.

Preferably, the first and second sections include hinged portions, and the movable joint comprises a hinge pivotally connecting the hinged portions for allowing the first and second sections to pivot between the operative and compact positions. The hinged portions are substantially coaxial in the operative position. The movable joint is also preferably configured for repeatably positioning the first and second sections in the operative position, which includes a discrete number of operative positions. More preferably, the discrete number of operative positions comprises only a single operative position.

In the compact position, the tool support device can preferably fit lying generally flat on an area measuring up to about 10 inches by 27 inches. In the operative position, however, the tool support is too large to fit within said space. More preferably, the tool support device in the compact position can lay flat within the space. In the compact position, the tool support device also is sufficiently compact to fit entirely within an autoclave tray space but is not sufficiently compact to fit an autoclave tray space when in the operative position.

The surgical tool support device preferably has a height in the operative position and also has a height in the compact position that is less than about 75% of the height in the operative position. The device also preferably includes an elbow that fixes together the first and second elongated portions. The first section of the device preferably includes another part of the second elongated portion and the elbow.

The tool mount of the surgical tool support device preferably includes a female threaded fastener portion that is configured to receive a fastener of a tool holder. More preferably, the tool mount is configured to releasably mount the surgical tool thereto. The device further includes a base locking nut that is in slidable association with the second elongated portion such that axial rotation thereabout is restricted or prevented. The base locking nut is configured for engaging a base clamp for preventing axial rotation thereof about the second elongated portion.

Preferably, the movable joint and locking member are configured such that the first and second sections and the locking member are maintained associated during movement between the operative and compact positions and in the operative and compact positions.

In the preferred embodiment, locking member preferably includes a sleeve that is in slidable association with the first and second sections for sliding between a locked position, for locking the hinge, and a release position, for releasing the hinge to allow the hinge to pivot. Preferably, the locking member is positioned over both the first and second sections in the locked position and over only one of the first and second sections in the release position.

Preferably, the locking member includes a bayonet fitting for releasably securing the locking member in the locked position. The locking member also preferably is configured for axial rotation about the hinge for releasably securing the locking member in the locked position.

The invention thus provides a simple surgical tool support device of integral construction that is contractible to fit within a standard sterilization tray such that the tray and the device therein can be placed in an autoclave or other sterilization equipment, and is expandable for use in a surgical procedure.

### BRIEF DESCRIPTION OF THE DRAWINGS

FIG. 1 is a side view of an embodiment of the tool support device in an operative position, constructed according to the present invention;

FIG. 2 is a perspective view thereof in a compact position;

FIG. 3 is a side view the tool support device with a tool support mounted thereon; and

FIG. 4 is a perspective view of the tool support device secured in a swinger clamp that is mounted to a support rail.

### DETAILED DESCRIPTION OF THE PREFERRED EMBODIMENTS

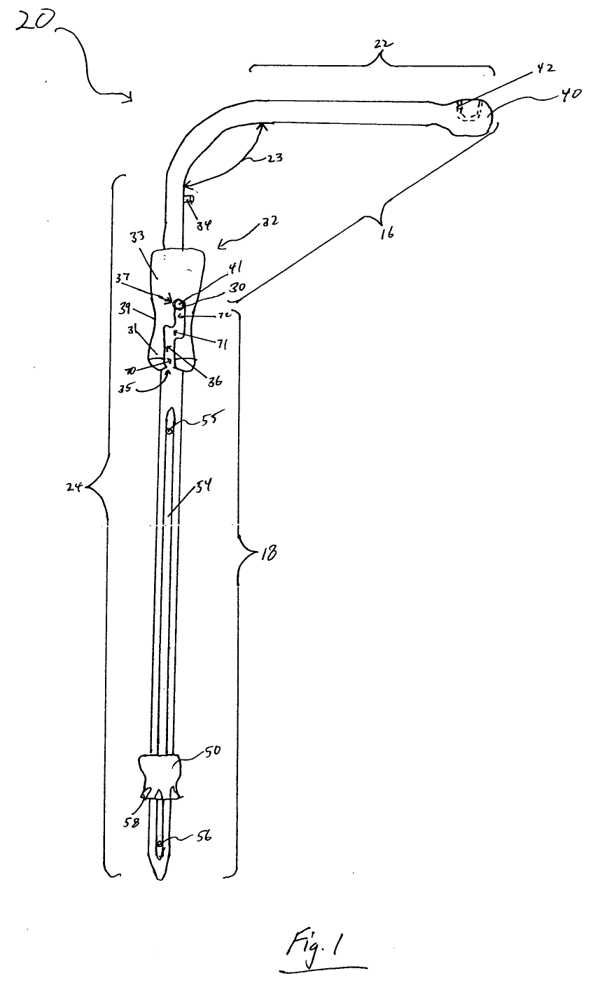

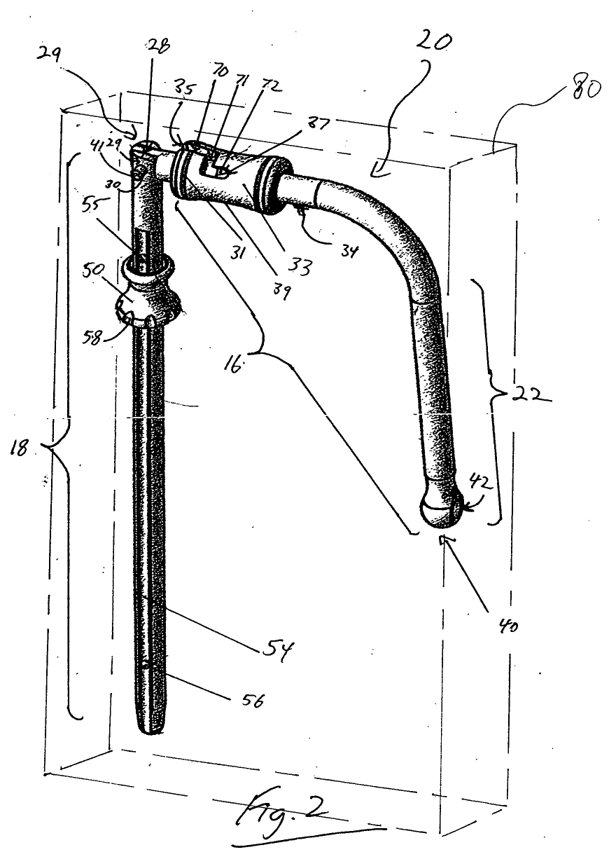

As shown in FIGS. 1 and 2, a preferred embodiment of the present invention includes a collapsible surgical tool support device 20, or bar, that includes a first elongated portion and a second elongated portion, or first and second legs. Preferably, the tool support device 20 is made of surgical grade stainless steel. Additionally, the first and second elongated portions, such as upper and lower elongated portions 22,24, preferably have a cylindrical configuration and have a minimum diameter of about 0.25 inches, and more preferably of about 0.5 inches. The upper and lower elongated portions 22,24 also preferably have a maximum diameter of about 1.5 inches, and more preferably of about 1 inch, and most preferably of about 0.75 inches. The preferred upper elongated portion 22, or bar, has a minimum length of about 4 inches, and more preferably of about 6 inches, and a maximum length of about 12 inches, and more preferably of about 10 or 8 inches. The preferred lower elongated portion 24 has a minimum length of about 12 inches, and more preferably of about 16 inches, and a maximum length of about 30 inches, and more preferably of about 26 inches. In one preferred embodiment, the tool support has an upper elongated portion 22 of 8.5 inches and a lower elongated portion 24 of 17 inches. In another preferred embodiment, the upper elongated portion 22 is 8.5 inches and the lower elongated portion 24 is 22 inches, and in yet another preferred embodiment, the upper elongated portion 22 is 8.5 inches and the lower elongated portion 24 is 26 inches. Alternative embodiments can be made of other suitable materials having alternative shapes and dimensions.

The upper and lower elongated portions 22,24 preferably have substantially straight configurations. In the operative position shown in FIG. 1, the upper and lower elongated portions 22,24 are in supportive association with each other and extend from each other at an angle 23. In one embodiment, the angle 23 is about 180°. In the preferred embodiment, the angle 23 is less than 180° and more preferably, is between about 150° and 50°. In this embodiment, the upper and lower elongated portions 22,24 are connected by an elbow or angle member that preferably has a substantially uniform curvature and the angle 23 between the upper elongated portion 22 and the lower elongated portion 24 is substantially about 90°. In other embodiments, the angle 23 is between about 100° and 70°.

The tool support device 20 is also divided into first and second movable sections, which in the embodiment shown in FIGS. 1 and 2 are upper and lower sections 16,18, since these are their relative positions during a typical surgical procedure. The upper section 16 preferably includes the upper elongated portion 22, and in the preferred embodiment, includes the upper elongated portion 22, the elbow, and an upper portion of the lower elongated portion 24. The lower section 18 preferably includes at least part of the lower elongated portion 24, and in this embodiment, includes the lower remainder thereof. One end of the upper section 16 is connected to one end of the lower section 18, although alternative connecting locations are possible.

The upper and lower sections 16,18 are movably, and preferably pivotally, connected to each other at a joint, and preferably at a hinge 26. The hinge 26 can include a tang 28 associated with one of the sections 16,18, such as the upper section 16, and a clevis 29 associated with the other of the sections 16,18, such as lower section 18, as shown in FIG. 2. Preferably, the tang 28 and clevis 29 extend from, and are of continuous and unitary construction with, respectively, the upper and lower sections 16,18 of the tool support device 20. In the preferred embodiment, the hinge 26 also includes a hinge pin 30 that extends through the tang 28 and clevis 29 such that the tang 28, and the upper section 16 extending therefrom, pivots about the axis of the hinge pin 30 with respect to the lower section 18. Alternative hinge embodiments can be used to pivotally connect the upper and lower sections 16,18. Also, other mechanisms can be employed to allow the sections 16,18 to move, and preferably pivot, with respect to each other.

The hinge 26 allows the upper and lower sections 16,18 to pivot between an operative position, shown in FIG. 1, and a compact position, shown in FIG. 2. In the operative position, the tool support device 20 is preferably fully extended and fixedly secured to an operating room table, and the lower elongated portion 24 can be placed in a substantially upright position so that the upper elongated portion 22 is substantially horizontal, preferably positioned over the patient. The surgical tools that depend from the upper elongated portion 22 are preferably in a position over a patient's body for practical use and manipulation by a physician. In other embodiments, the tool support device 20 can be positioned such that the upper elongated portion 22 and the surgical tools attached thereto are not over the patient.

Preferably, the hinge 26 is configured for repeatably positioning the upper and lower sections 16,18 in the operative position. More preferably, the operative position includes a predetermined and discrete number of operative positions. For example, one embodiment can include three predetermined and discrete positions where the upper and lower sections 16,18 are positioned, respectively, at angles of about 150°, 120°, and 90° from each other. Advantageously, the tool support device 20 having predetermined and discrete positions allows the device, and the surgical tools mounted thereto, to be returned to precisely the same positions after adjustment or cleaning thereof. In the preferred embodiment shown in FIG. 1, the operative position includes only a single operative position in which the upper section 16 is positioned at an angle of substantially about 180° with respect to the lower section 18.

When the tool support device 20 is in the compact position, the planar and volumetric space that the device occupies is significantly reduced. In this position, the tool support device 20 preferably can advantageously fit within a standard sterilization tray, whereas in the operative position, the tool support device 20 is typically too large to fit in the same tray. Preferably, the height of the tool support device 20 in the compact position is less than about 75% of the height of the tool support device in the operative position. More preferably, the height in the compact position is less than about 66%, and even more preferably less than about 50%, of the height of the device in the operative position.

Preferably, the upper section 16 pivots about the axis of the hinge pin 30 such that in a compact position, the upper section 16 is at an angle of about 90° with respect to the lower section 18. In other embodiments, the upper section 16 can be at other angles with respect to the lower section 18 in the compact position while still occupying a significantly reduced space compared to its configuration in the operative position.

With the tool support device 20 in the compact position, the tray and the tool support device 20 therein can be inserted into an autoclave for sterilization. Preferably, the tool support device 20 in the compact position can fit within a sterilization tray preferably having a minimum width of about 4 inches and more preferably of about 6 inches, and a maximum width preferably of about 20 inches and more preferably of about 12 inches. The minimum length of the tray is preferably about 15 inches and more preferably about 20 inches, and the maximum length of the tray is preferably about 40 inches and more preferably about 30 inches. The minimum height of the tray is preferably about 2 inches and more preferably about 4 inches, and the maximum height of the tray is preferably about 10 inches and more preferably about 6 inches. In the preferred embodiment, the tool support device 20 in the compact position can fit within a tray that is at least about 10 inches wide, 27 inches long, and 4 inches tall. The tool support device 20 in the compact position can also lay substantially flat within a standard sterilization tray, without having to be skewed diagonally or inclined against one of the vertical walls of the tray 80, as shown in FIG. 2. Advantageously, the single piece construction of the tool support device 20 eliminates the need to disassemble the device when inserting it into the sterilization tray, thus removing the risk that any single piece thereof may be lost, misplaced, or misassembled during use in the next surgical procedure.

The tool support device 20 also includes a locking mechanism 32 that is associated with the hinge 26 for locking the hinge 26 in the operative position and releasing it therefrom. Advantageously, when the locking mechanism 32 is in the locked position shown in FIG. 1, the locking mechanism 32 locks the hinge 26 in the operative position with sufficient strength and rigidity such that the upper elongated portion 22 and any surgical tool, including surgical tool supports, mounted thereto is substantially rigidly supported by the lower section 18. While some play is acceptable with the locking association, the locking association is sufficient for supporting torsional, twisting, and other loads imposed thereon by the surgical tools during surgery. Preferably, the locking mechanism 32 includes a locking member 31, such as a sleeve 33 that slides over the hinged portions of the upper and lower sections 16,18 and the hinge 26.

When the locking member 31 is in the locked position shown in FIG. 1, the locking member 31 is simultaneously disposed over both ends of the upper and lower sections 16,18 and the hinge 26. In this position, the locking member 31 prevents the hinge 26 from pivoting to substantially fix the tool support device 20 in the operative position. When the locking member 31 is slid to the release position, the locking member 31 is only disposed over one of the upper section 16 or the lower section 18. In the preferred embodiment, the locking member 31 is a sleeve 33 that is disposed over the upper section 16 in the release position, as shown in FIG. 2. In the release position, the locking member 31 allows the upper and lower sections 16,18 to pivot about the hinge 26 and thus the tool support device 20 can pivot to and from the compact position.

The locking member 31 preferably has a minimum outer diameter of about 1 inch and maximum outer diameter of about 2 inches. More preferably, the outer diameter of the locking member is around 1.25 inches. The locking member 31 preferably has a sufficient length such that it can slide over and simultaneously cover both ends of the upper and lower sections 16,18 sufficiently to block the hinge 26 from pivoting from the operative position. The locking member 31 preferably has a minimum length of about 2 inches and maximum length of about 3 inches. More preferably, the length of the locking member 31 is about 2.5 inches. Additionally, the ratio of the axial length of the locking member 31 to the inner diameter thereof is preferably between about 2:1 and 6:1, and more preferably about 4:1. In the preferred embodiment, the exterior surface of the sleeve 33 has an indentation 39 to facilitate grasping and sliding by a user, preferably giving the sleeve 33 an hour glass shape. Advantageously, the shape and dimensions of the sleeve 33 is readily conformable to human grip, which allows the sleeve 33 to be easily manipulated between the locked and release positions.

In the preferred embodiment, the locking member 31 is configured to freely rotate axially about the upper section 16 when the locking member 31 is in the release position. The upper section 16 preferably includes a stopping pin 34 that protrudes from the surface thereof. The stopping pin 34 is configured to provide a ceiling for the locking member 31 along the upper section 16 past which the locking member 31 cannot slide.

Additionally, the locking mechanism 32 of the preferred embodiment preferably includes a bayonet pin 41 protruding from one of the upper or lower sections 16,18, preferably the lower section 18, and a bayonet slot 36 defined in the sleeve 33. In an alternate embodiment, the pin can be on the sleeve 33 with the corresponding slot on one of the sections 16,18. Preferably, the bayonet pin 41 is coaxial with the hinge 26, and in the preferred embodiment, the pin 41 is of integral construction with the hinge pin 30 and preferably protrudes from the surface of one side of the clevis 29 that extends from the lower section 18. Other positions and locations for the bayonet pin 41 can also be used.

In the preferred embodiment, slot 36 is configured for receiving the bayonet pin 41 as the sleeve 33 slides into the locked position. Preferably, opening 35 of the slot 36 is disposed at the leading end of sleeve 33 as the sleeve slides in the locked position, with the leading end facing the lower section 18. The slot 36 preferably has an outer portion 70 that extends generally axially from the opening 35 to partway up the length of the sleeve 33. A mid-portion 71 of the slot 36 extends generally circumferentially to require the sleeve 33 to be rotated about its axis when receiving the pin. An inner portion 72 of the slot 36 extends generally axially further up the length of the sleeve 33 to allow the sleeve to slide generally axially along the pin 41 to the locked position. The inner portion 72 terminates at an end portion 37. As shown in FIG. 1, the outer, mid, and inner portions of the slot 36 are oriented at angles to each other within about 20°, and more preferably within about 10° or 5°, of a right angle.

As the sleeve 33 slides over the hinge 26 to the locked position, the sleeve 33 is preferably axially rotated about the upper and lower sections 16,18 such that opening 35 of the slot 36 is aligned with the protruding pin 41. The sleeve 33 can then continue to slide to the locked position as the slot 36 receives the pin 41 therein. The sleeve 33 preferably reaches and is secured in the locked position when the pin 41 contacts the end portion 37 of the slot 36, as shown in FIG. 1. Thus, the association between the pin 41 and the slot 36 advantageously provides a bottom limit for the sleeve 33 past which the sleeve cannot slide. When used in the preferred upright position shown in FIG. 1, gravity retains the sleeve 33 in the locked position, and the mid-portion 71 of the slot 36 prevents the sleeve 33 from being accidentally knocked out of this position.

In the preferred embodiment shown in FIG. 1, the slot 36 of the sleeve 33 has an S-shape configuration. Thus, as the pin 41 is received in the slot 36 and reaches the junction between the outer portion 70 and the mid-portion 71, the sleeve 33 must be rotated axially about the upper and lower sections 16,18 to allow the sleeve 33 to continue to slide until the pin 41 reaches the end portion 37 of the slot 36 in the locked position. The S-shape configuration of the slot 36 advantageously provides a more secure and rigid support for the locking member 31 in the locked position. Also, the slot 36 can extend completely or partially through the wall thickness of the sleeve 33.

In other embodiments, the locking member 31 can include other mechanisms for releasably locking the hinge in the operative position. For example, a locking pin or latch can be received in both the upper and lower sections 16,18 to fix the sections against rotation or movement from the locked position.

Referring FIGS. 1, 2, and 3, the end of the upper elongated portion 22 that is opposite the lower elongated portion 24 includes a tool mount 40 that is configured for mounting a surgical tool or instrument thereto. The term “surgical tool,” as used herein, should generally be understood to include tools or instruments, such as a laparoscope, endoscope, retractor, forcep, clamp, scalpel, or the like, used during surgical procedures that can be mounted to the tool support device 20, and also tool supports that can be mounted to the tool support device 20 from which other surgical tools or instruments can be mounted or otherwise attached. Preferably, the tool mount 40 includes a female threaded portion 42 or bore, that is configured for fixedly and securingly receiving a complimentary male screw portion of a surgical tool holder or other attachment device. The tool holder is preferably a clamp configured for holding or securing a surgical tool therein. In another embodiment, the tool holder can attach or clamp directly onto the upper elongated portion 22 instead of screwing into the tool mount 40. In other embodiments, the tool mount 40 can include other attachment devices for releasably mounting tools thereto.

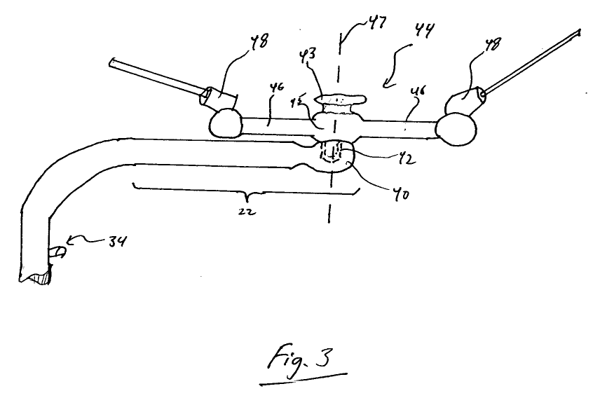

In one embodiment shown in FIG. 3, the tool mount 40 is configured to receive a hydra tool support 44 that includes a yoke 45 and opposing fixed arms 46 that extend from the yoke 45. Preferably, the yoke 45 includes a fastener 43 that is complimentary to, and configured to be received in, the female threaded portion 42 of the tool mount 40 for fixedly securing the tool support 44 in the tool mount 40 of the upper elongated portion 22. Advantageously, the tool support 44 is configured such that the yoke 45 can rotate about the axis 47 that is substantially orthogonal to the upper elongated portion 22 before fastening. When the desired orientation of the tool support 44 is achieved, the fastener 43 is screwed into the female threaded portion 42 to fixedly secure the tool support 44 on the tool mount 40.

Disposed at the ends of the opposing arms 46 are rotatable swivel attachments 48, to which a series of other surgical tools or instruments can attach. Preferably, the swivel attachments 48 can be locked in place as known in the art to support and maintain surgical tools attached thereto in a stable position. Advantageously, the tool support 44 and the tools and other equipment that are attached thereto allow a physician great flexibility in positioning the surgical tools in the desired position and orientation necessary to perform their function during a surgical procedure.

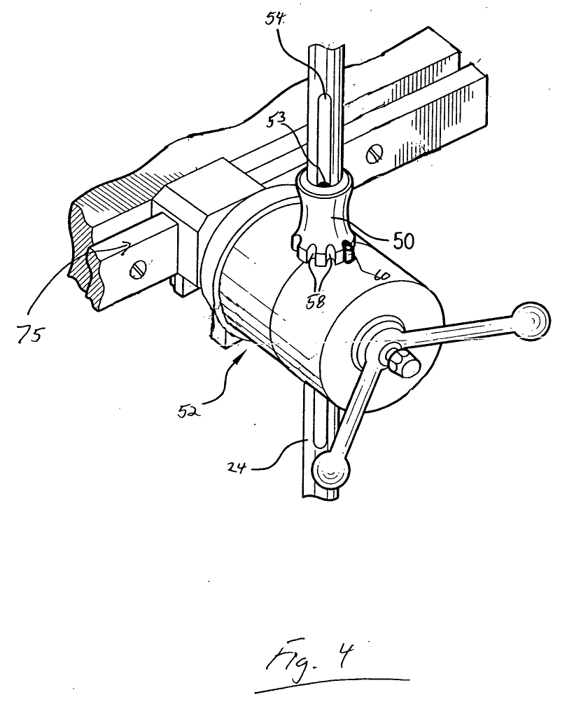

Referring to FIGS. 1 and 4, the other end of the lower elongated portion 24, which is disposed opposite the upper elongated portion 22, is configured for adjustably and fixably securing the lower elongated portion 24 to the operating room table such that the tool support device 20 is stably supported thereon. Preferably, the lower elongated portion 24 includes a base locking nut 50 or other sleeve member that is configured for associating with a base member or base clamp 52, for example a swinger clamp as disclosed in U.S. Pat. No. 6,315,260, to allow an angular orientation to be selected and retained with respect to the clamp 52.

The base locking nut 50 preferably comprises a sleeve in slidable association with the lower elongated portion 24. Similar to the sleeve 33, the exterior surface of the base locking nut 50 can have an hour glass shape to facilitate human grip and to allow the base locking nut 50 to be easily manipulated over the lower elongated portion 24. In the preferred embodiment, the base locking nut 50 includes a guide key 53 or wedge on its inner annular surface. The key guide 53 is configured for reception within a groove 54 that runs along one side of the lower elongated portion 24. Preferably, the groove 54 runs from the adjacent and lower end of the lower elongated portion 24 that is opposite the upper elongated portion 22 to at least about halfway up the length of the lower elongated portion 24, and can extend almost completely along the length of the lower section 18, preferably ending below the locking mechanism 32 in the locked position. The guide key 53 and groove 54 are preferably configured to limit or prevent the axial rotation of the base locking nut 50 about the lower elongated portion 24 as the base locking nut 50 slides thereover. In the preferred embodiment, the lower elongated portion 24 also preferably includes first and second stopping pins or knobs 55 and 56 that can be disposed in the groove 54 and located near each end of the groove. The stopping pins 55,56 are disposed to block axial movement when they abut part of the base locking nut 50, such as the guide key 53, which prevents the base locking nut 50 from sliding past the stopping pins.

In other embodiments, the association between the lower elongated portion 24 and the base locking nut 50 can include different configurations for preventing the base locking nut 50 from freely rotating about the lower elongated portion 24. For example, the lower elongated portion 24 can include a track that protrudes from the surface thereof and runs along the length of the lower elongated portion 24. The base locking nut 50 would have a complimentary receiving indentation configured to allow the base locking nut 50 to ride along the track while preventing the axial rotation of the base locking nut 50 about the lower elongated portion 24.

As shown in FIG. 4, the base locking nut 50 is configured for associating with the base clamp 52 or holder block. Preferably, the base locking nut 50 includes a series of notches 58 that are disposed around the lower circumference or collar of the base locking nut 50. The notches 58 are configured for engaging a protrusion 60 of the base clamp 52 to maintain and secure the rotational orientation between the tool support device 20 and the base clamp 52. The restriction in rotational movement of the lower elongated portion 24 advantageously helps maintain the stable positioning of the surgical tools that are mounted to the upper elongated portion 22 of the tool support device 20. If it is necessary to adjust the tool support device 20 by axially rotating the lower elongated portion 24, the base locking nut 50 is simply disengaged from the base clamp 52 and slid up the length of the lower elongated portion 24. The lower elongated portion 24 can then be axially rotated to a desired position, and then the base locking nut 50 can again engage the base clamp 52 such that the protrusion 60 fixedly associates with an adjacent or otherwise neighbouring notch 58 about the circumference of the base locking nut 50.

Preferably, the base clamp 52 also fixedly secures the lower elongated portion 24 and the rest of the tool support device 20 to the operating room table, such as by clamping to a rail 75 thereof, and provides sufficient strength and rigidity to the tool support device 20 to stably mount surgical tools thereto.

The term “about,” as used herein, should generally be understood to refer to both the corresponding number and a range of numbers. Moreover, all numerical ranges herein should be understood to include each whole integer within the range.

While illustrative embodiments of the invention are disclosed herein, it will be appreciated that numerous modifications and other embodiments can be devised by those of ordinary skill in the art. Features of the embodiments described herein can be combined, separated, interchanged, and/or rearranged to generate other embodiments. Therefore, it will be understood that the appended claims are intended to cover all such modifications and embodiments that come within the spirit and scope of the present invention.

What is claimed is:

1. A surgical tool support, comprising:
a first elongated portion comprising a tool mount that is configured to mount a surgical tool thereto;
a second elongated portion in supportive association with the first elongated portion and extending therefrom at a first angle of less than 180°, wherein the second elongated portion is configured for adjustably and fixably being secured to a base member for supporting the tool support, wherein the tool support comprises:
a first section, which includes the first elongated portion, and
a second section, which includes at least part of the second elongated portion;
a moveable joint that movably connects the first and second sections of the tool support for movement between:
an operative position, and
a compact position, in which the first and second sections are oriented such that the tool support is substantially more compact than in the operative position; and
a locking member associated with the movable joint for releasably locking the movable joint in the operative position with sufficient strength and rigidity such that first elongated portion and any surgical tool mounted thereto is substantially rigidly supported by the second section.

2. The surgical tool support of claim 1, wherein the first and second sections comprise hinged portions, and the movable joint comprises a hinge pivotally connecting the hinged portions for allowing the first and second sections to pivot between the operative and compact positions, wherein the hinged portions are substantially coaxial in the operative position.

3. The surgical tool support of claim 1, wherein the movable joint is configured for repeatably positioning the first and second sections in the operative position, which includes a discrete number of operative positions.

4. The surgical tool support of claim 3, wherein the discrete number of operative positions comprises only a single operative position.

5. The surgical tool support of claim 1, wherein, in the compact position, the tool support can fit lying generally flat on an area measuring up to about 10 inches by 27 inches, but in the operative position, the tool support is too large to fit within said space.

6. The surgical tool support of claim 5, wherein, in the compact position, the tool support can lay flat within the space.

7. The surgical tool support of claim 1, wherein the tool support has a height in the operative position and a height in the compact position that is less than about 75% of the height in the operative position.

8. The surgical tool support of claim 1, further comprising an elbow fixing together the first and second elongated portions.

9. The surgical tool support of claim 8, wherein the first section comprises another part of the second elongated portion and the elbow.

10. The surgical tool support of claim 1, wherein the first angle is between about 70° and 110°.

11. The surgical tool support of claim 1, wherein the tool mount comprises a female threaded fastener portion that is configured to receive a fastener of a tool holder.

12. The surgical tool support of claim 1, further comprising a base locking nut in slidable association with the second elongated portion such that axial rotation thereabout is restricted or prevented, the base locking nut being configured for engaging a base clamp for preventing axial rotation thereof about the second elongated portion.

13. The surgical tool support of claim 1, wherein the tool mount is configured to releasably mount the surgical tool thereto.

14. The surgical tool support of claim 1, wherein the surgical tool support in the compact position is sufficiently compact to fit entirely within an autoclave tray space but is not sufficiently compact to fit an autoclave tray space when in the operative position.

15. The surgical tool support of claim 1, wherein the movable joint and locking member are configured such that the first and second sections and the locking member are maintained associated during movement between the operative and compact positions and in the operative and compact positions.

16. A surgical tool support, comprising:
a first elongated portion comprising a tool mount that is configured to mount a surgical tool thereto;
a second elongated portion in supportive association with the first elongated portion and extending therefrom at a first angle, wherein the second elongated portion is configured for adjustably and fixably being secured to a base member for supporting the tool support, wherein the tool support comprises:
a first section, which includes the first elongated portion, and
a second section, which includes at least part of the second elongated portion;
a hinge that pivotally connects the first and second sections of the tool support for movement between:
an operative position, and
a compact position, in which the first and second sections are oriented such that the tool support is substantially more compact than in the operative position; and
a locking member associated with the movable joint for releasably locking the hinge in the operative position with sufficient strength and rigidity such that first elongated portion and any surgical tool mounted thereto is substantially rigidly supported by the second section.

17. The surgical tool support of claim 16, wherein the locking member comprises a sleeve in slidable association with the first and second sections for sliding between a locked position, for locking the hinge, and a release position, for releasing the hinge to allow the hinge to pivot.

18. The surgical tool support of claim 17, wherein the locking member is positioned:
over both the first and second sections in the locked position; and
over only one of the first and second sections in the release position.

19. The surgical tool support of claim 18, wherein the locking member comprises a bayonet fitting for releasably securing the locking member in the locked position.

20. The surgical tool support of claim 17, wherein the locking member is configured for axial rotation about the hinge for releasably securing the locking member in the locked position.

21. A surgical tool support, comprising:
a first elongated portion comprising a tool mount that is configured to mount a surgical tool thereto;
a second elongated portion in supportive association with the first elongated portion and extending therefrom at a first angle, wherein the second elongated portion is configured for adjustably and fixably being secured to a base member for supporting the tool support, wherein the tool support comprises:
a first section, which includes the first elongated portion, and
a second section, which includes at least part of the second elongated portion;
a moveable joint that movably connects the first and second sections of the tool support for movement between:
a discrete number of operative positions, in which the first and second sections are repeatably positionable, and
a compact position, in which the first and second sections are oriented such that the tool support is substantially more compact than in the operative position; and
a locking member associated with the movable joint for releasably locking the movable joint in the operative position with sufficient strength and rigidity such that first elongated portion and any surgical tool mounted thereto is substantially rigidly supported by the second section.

22. The surgical tool support of claim 21, wherein the movable joint comprises a hinge that pivotally connects the first and second sections for pivoting between the operative and compact positions.

23. The surgical tool support of claim 22, wherein the discrete number of operative positions comprises only a single operative position.
